(19) United States
(12) Patent Application Publication
(10) Pub. No.: US 2012/0065712 A1
(43) Pub. Date: Mar. 15, 2012

(54) CELLULAR STIMULATION BY OPTICAL ENERGY

(75) Inventors: Rudolfo L. Rivera, Plano, TX (US); Richard T. Rivera, Dallas, TX (US)

(73) Assignee: INTELLECTUAL RESOURCES LLC, Dallas, TX (US)

(21) Appl. No.: 13/231,849

(22) Filed: Sep. 13, 2011

Related U.S. Application Data
(60) Provisional application No. 61/382,440, filed on Sep. 13, 2010.

(51) Int. Cl. A61N 5/067

(52) U.S. Cl. 607/89

(57) ABSTRACT

An apparatus and associated method for a therapeutic laser device having a laser source operably generating coherent optical energy radiation having a wavelength in a range of about 950 nanometers to about 1,200 nanometers and at a power output from a treatment head in a range from about 0.05 watts per square centimeter to about 2.0 watts per square centimeter.

### RELATED APPLICATION

This application claims the benefit of the earlier filing date of U.S. Provisional Patent Application Ser. No. 61/382,440.

### SUMMARY

Some embodiments of the present invention contemplate an apparatus for therapeutically treating living tissue. The apparatus includes a laser source operably generating coherent optical energy radiation having a wavelength in a range of about 950 nanometers to about 1,200 nanometers. An optics connector is connected at a proximal end in optical communication with the laser source. A treatment head is connected to a distal end of the optics connector. The treatment head has an optical arrangement operably focusing the coherent optical energy radiation into a light beam defining a cross-sectional area in a range from about 12 square centimeters to about 20 square centimeters where the light beam emanates from the treatment head.

Some embodiments of the present invention contemplate a therapeutic laser device having a laser source operably generating coherent optical energy radiation having a wavelength in a range of about 950 nanometers to about 1,200 nanometers and at a power output from a treatment head in a range from about 0.05 watts per square centimeter to about 2.0 watts per square centimeter.

Some embodiments of the present invention contemplate a method including steps of: obtaining an apparatus having a laser source operably generating coherent optical energy radiation with a wavelength in a range of about 950 nanometers to about 1,200 nanometers, an optics connector connected at a proximal end in optical communication with the laser source, and one or more treatment heads each connected to a distal end of a respective one of the optics connector, each treatment head operably focusing the coherent optical energy radiation into a light beam defining a cross-sectional area in a range from about 12 square centimeters to about 20 square centimeters where the light beam emanates from the treatment head; aiming the treatment head to irradiate a selected live tissue workpiece with the coherent optical energy radiation; and controlling a dwell time that the selected live tissue workpiece is irradiated in accordance with a predefined treatment protocol.

### BRIEF DESCRIPTION OF THE DRAWINGS

FIG. 1 is an isometric depiction of a therapeutic laser apparatus that is constructed in accordance with embodiments of the present invention.

FIG. 2 is another isometric depiction similar to FIG. 1.

FIG. 3 is a flowchart of steps in a method for AUTOCALIBRATION in accordance with embodiments of the present invention.

FIG. 4 is an isometric depiction of the right-hand side of the apparatus of FIG. 1 with a portion of the enclosure removed.

FIG. 5 is an isometric depiction of the left-hand side of the apparatus of FIG. 1 with another portion of the enclosure removed.

### DETAILED DESCRIPTION

Embodiments of the present invention are directed to a therapeutic laser treatment apparatus and associated method for its use. The novel construction and capabilities of the present embodiments make it possible to deliver volumetric effect dosages of optical energy to living tissue being treated by way of relatively high power and large-scale delivery of the optical energy for purposes of the treatment. The present embodiments solve problems known to exist in previous attempted solutions that cannot reliably transmit enough optical energy in a reasonable time through melanin and through blood and water barriers of living tissue to effect meaningful and positive biochemical and cellular treatment deep into the body. The advantageous benefits of the volumetric effect dosages are achievable without risk that the comparatively higher energy density (W/cm²) heats the living tissue too fast or too much such that ablation occurs.

In accordance with these embodiments, particularly characteristic optical energy generated by a laser is advantageously generated and supplied to the living tissue for various treatment purposes. The monochromatic and coherent nature of laser light is absorbed by the living tissue in relation to the particular characteristics of the optical energy and in relation to certain properties of the irradiated living tissue.

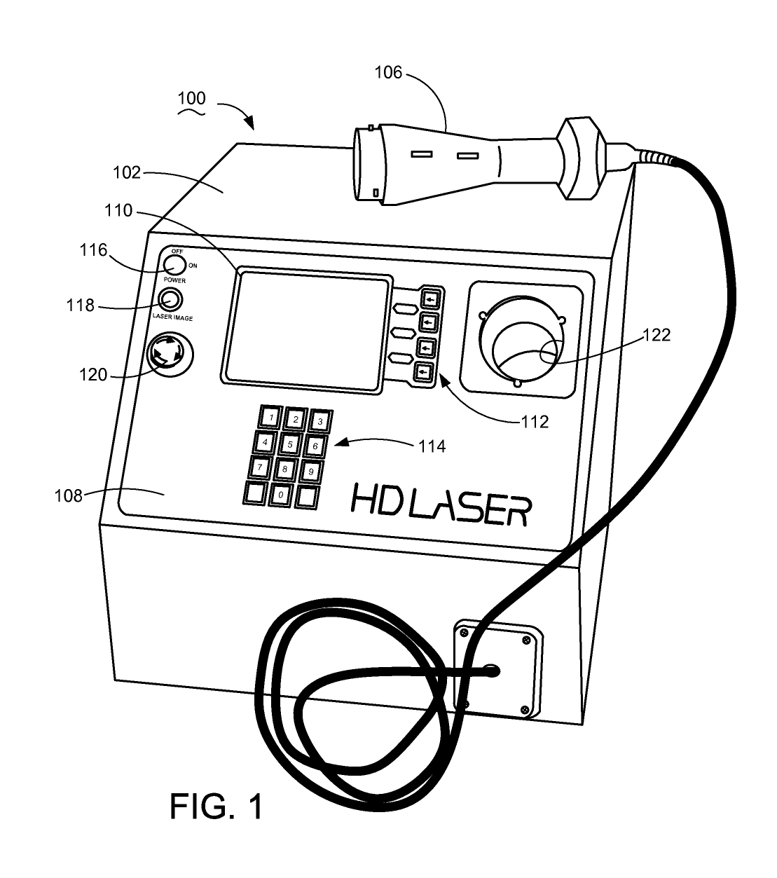

FIG. 1 is an isometric depiction of a therapeutic laser 100 constructed and used in accordance with the claimed embodiments to advantageously treat living tissue (not depicted). As will be appreciated the living tissue being treated can be a diverse variety of things like skin, muscle, organs, and the like, so the living tissues contemplated by the disclosed embodiments are collectively characterized by the term “living tissue workpiece” because no specific enumeration is necessary for the skilled artisan to readily ascertain the scope of the claimed embodiments in those terms.

The therapeutic laser 100 is generally contained within an enclosure 102 that protects the internal components while exposing all the necessary controls that a user needs for operation of the device. The enclosure 102 is preferably removable from an underlying frame structure to gain access to the internal components as need be for servicing or repair. As discussed above, one or multiple interlock devices, such as a mechanical switch or a proximity switch or the like, is preferably supported on the framework and actuated by the enclosure to disable the device whenever the protective enclosure 102 is removed.

Generally, extending from the enclosure 102 is a proximal end of an optics connector 104, such as a fiber optics guide, and a treatment head 106 is connected to a distal end of the optics connector 104. The flexibility of the optics connector 104 advantageously makes the treatment head 106 selectively moveable in relation to the enclosure 102 and its contents, such as the laser source. The length of the optics connector 104 is selected to accommodate the distance from where the therapeutic laser 100 is located, such as a shelf or cart, and the living tissue workpiece. The treatment head 106 is sized to be readily adapted for hand-held manipulation in treating the living tissue workpiece. In other embodiments, the treatment head 106 can be robotically manipulated for computer-assisted control of the treatment head 106 movements during a treatment protocol.

The enclosure 102 has an opening surrounding a control panel 108 that supports a number of controls. On-board software is automatically executed when the therapeutic laser 100 is powered on, which initializes the equipment and then provides a menu tree of prompts to the user via a graphical interface such as the liquid crystal display 110 depicted in these embodiments. A number of depressable selection buttons 112 are provided for the user to make menu responses, and a numeric keypad 114 is provided for the user to enter other requested input such as a selected power level and the like.

A key operated switch 116 provides a top level shutdown of all components of the therapeutic laser 100 to ensure no unauthorized usage. An illuminating indicator 118 signals whenever the laser source is generating coherent optical energy radiation. A push-pull palm button 120 provides an emergency stop for immediately powering down the laser source.

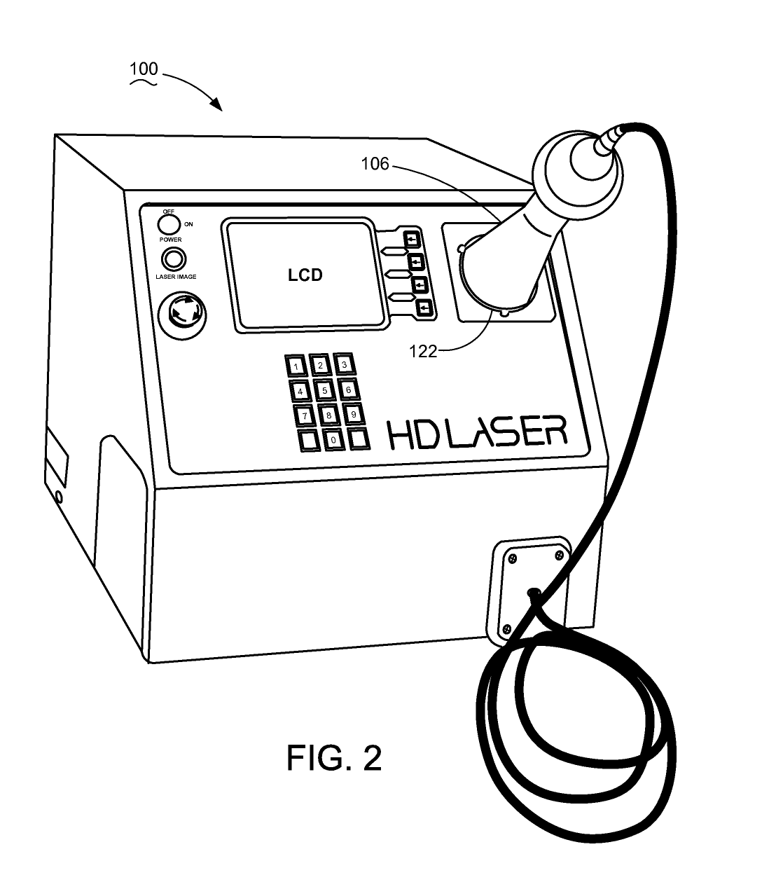

A cradle 122 is formed by an aperture that is sized to receivingly engage a distal end of the treatment head 106. FIG. 1 depicts the treatment head 106 removed from the cradle 122 such as it would be during a treatment procedure. FIG. 2 is another isometric depiction similar to FIG. 1 but alternatively depicting the treatment head 106 stored away in the cradle 122 such as it would be during idle times between treatment procedures. One of the interlock devices, such as a mechanical switch or a proximity switch or the like, is supported by the cradle to indicate whenever the treatment head 106 is disposed in the cradle. A power meter is located inside the enclosure 102 in optical communication with the treatment head 106 when the treatment head is disposed in the cradle 122.

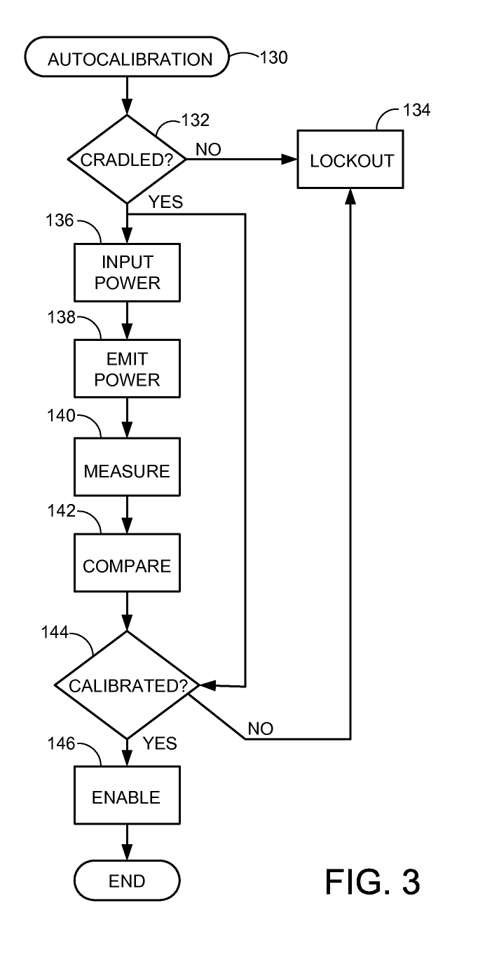

The on-board software includes calibration logic that requires the treatment head 106 be calibrated in regard to power level of the emitted optical radiation before the therapeutic laser 100 is made ready for usage in a treatment. Preferably, the calibration logic requires that calibration be performed before each and every usage of the therapeutic laser 100. For example, FIG. 3 is a flowchart depicting steps in a method 130 for AUTOCALIBRATION in accordance with embodiments of the present invention.

The method 130 begins in block 132 with a determination as to whether or not the treatment head is in the cradle as is depicted in FIG. 2. Verification that the treatment head is in the cradle is provided by monitoring the signal from the interlock on the cradle. As previously discussed, when the treatment head is in the cradle its output end is placed in optical communication with a power meter inside the enclosure. If the determination of block 132 is “no,” then the therapeutic laser is locked out from operation (in lockout mode) in block 134. Otherwise, control passes to block 136 where the therapeutic laser prompts the user to input a desired power level of the optical radiation from the laser source. That user input can be performed by pressing one of multiple offered selections via the pressable buttons on the control panel or by entering a numeric value via the keypad on the control panel. Note that if the determination of block 132 becomes “no” during the operation of block 136 (indicating the treatment head has been removed from the cradle) then the therapeutic laser goes into lockout mode in block 134.

In block 138 the laser source inside the enclosure is adjusted in response to the selected power level input in block 136, and the laser source is thus enabled to communicate the optical radiation to the power meter in the enclosure. Again, if the determination of block 132 becomes “no” during the operation of block 138 then the therapeutic laser goes into lockout mode (including disabling the laser source) in block 134.

In block 140 the optical radiation is measured by the power meter in the cradle, and in block 142 that measured value is compared to a threshold value associated with the selected power level that was input by the operator in block 136. For example, the threshold value can be the selected power level itself, or it can be a marginal value calculated from the selected power level itself. As above, if the determination of block 132 becomes “no” during the operation of block 142 then the therapeutic laser goes into lockout mode in block 134.

Block 143 makes a determination as to whether the laser source is in within a required calibration parameter based on the comparison of the measured and threshold values in block 142. For example, if the difference between the measured value and the threshold value is less than a predetermined allowed variation, either based on a quantity or a percentage difference, then the calibration logic deems the laser source to be within calibration. That is, if the predetermined allowed variation is 0.4 Watts, the selected power level is 15 Watts, and the measured value is 14.8 Watts, then in that case the determination of block 144 is “yes.” Again, if the determination of block 132 becomes “no” during the operation of block 144 then the therapeutic laser goes into lockout mode in block 134.

If the determination of block 144 is “yes,” then the calibration logic enables the laser at the selected power level in block 146, permitting the user to remove the treatment head from the cradle for usage at the selected power level for treatment of the living tissue workpiece. Importantly, the control system will not permit the user to change the selected power level without first returning the treatment head to the cradle and performing the AUTOCALIBRATION method 130 over for the newly selected power level. Further, if the laser source is shut down by any means while the treatment head is removed from the cradle, then the control system will require that the AUTOCALIBRATION method 130 be performed before again enabling the laser source.

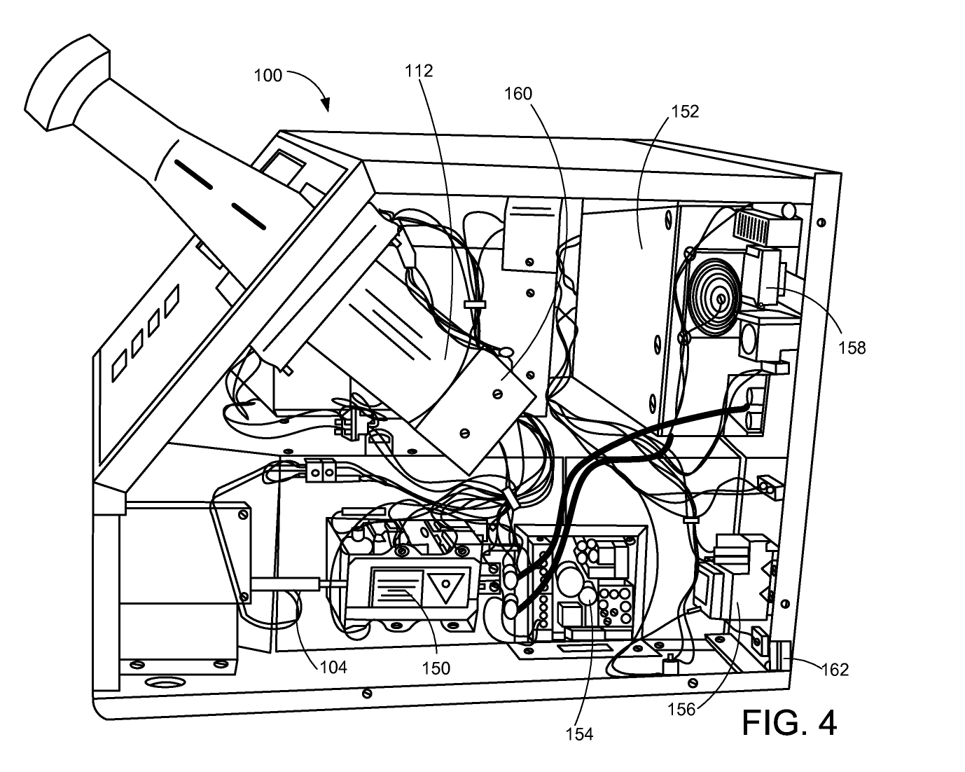

FIG. 4 is yet another isometric depiction of the therapeutic laser 100 with a right-side portion of the enclosure removed to reveal some of the internal components. A laser diode module (“laser source”) 150 selectively communicates coherent optical energy radiation to the optics connector 104. Embodiments of the present invention contemplate the laser source 150 generally being capable of generating coherent optical energy radiation having a wavelength in a range of about 950 nanometers to about 1,200 nanometers. Preferably, the laser source 150 is capable of generating the coherent optical energy radiation having a wavelength in a range of about 1,000 nanometers to about 1,150 nanometers, operating at a primary wavelength of substantially 1,064 nanometers.

Working power is provided to the laser source 150 by a power supply module 152. Generally, the power level of the coherent optical energy radiation is selectable to a power level in a range from about 10 Watts to about 100 Watts. Preferably, the laser source 150 is selectable by the user to provide the coherent optical energy radiation at a maximum power level of about 20 Watts. A thermoelectric temperature controller 154 maintains the laser source 150 at or below a specified working temperature. An inlet supply power receptacle 156 transmits external power to the therapeutic laser 100. A control voltage power supply 158 provides low voltage to the control components. The cradle 112 supports the power meter 160 for use as described above in the AUTOCALIBRATION method 130. An interlock switch 162 indicates whether the side portion of the enclosure is attached, with the control system placing the therapeutic laser 100 in lockout mode if the side portion of the enclosure is removed as in this depiction.

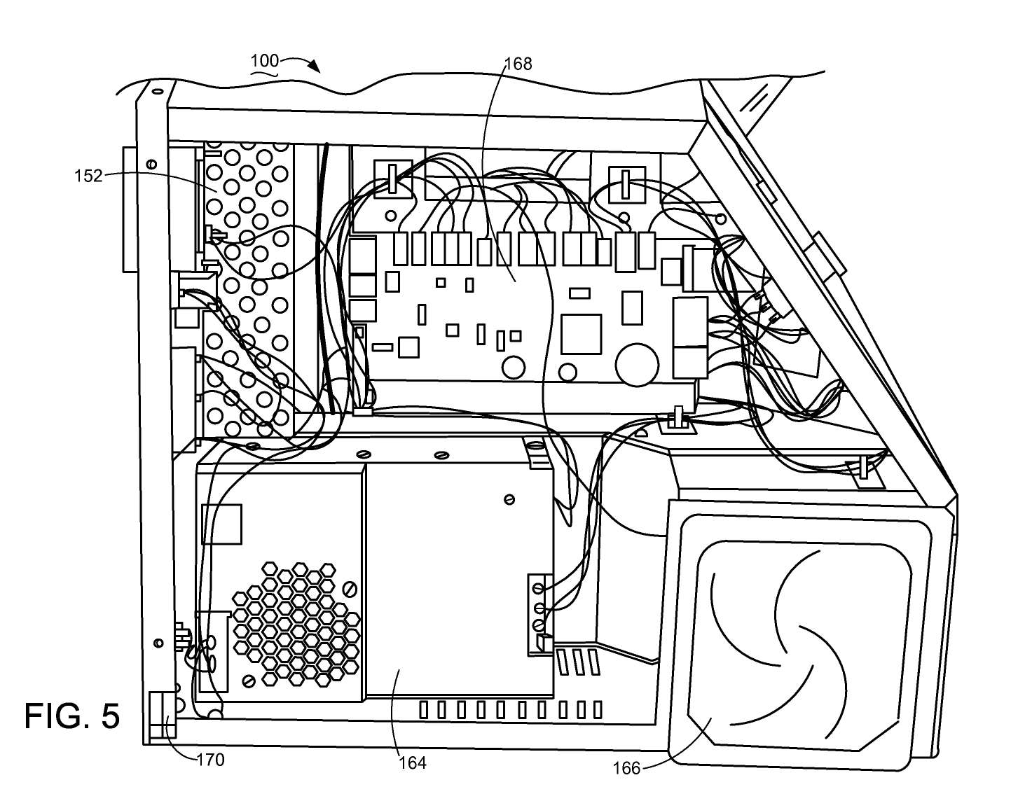

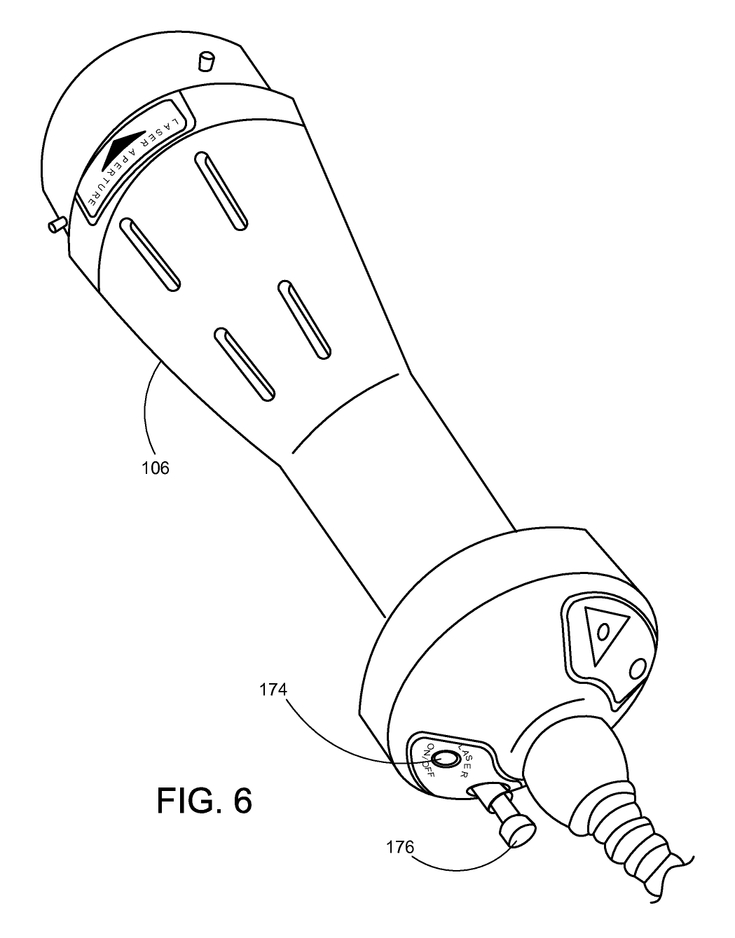

FIG. 5 is a view similar to FIG. 4 but showing the opposing portion of the enclosure removed to reveal more of the internal components. Power to the thermoelectric cooler is provided by a power supply module 164. A cooling air exhaust 166 draws cooling air through the therapeutic laser 100. A main control board 168 is where most of the components of the top level control system reside. Another interlock switch 170, like the interlock switch 162, indicates whether the left-side portion of the enclosure is attached, with the control system placing the therapeutic laser 100 in lockout mode if the left-side portion of the enclosure is removed as in this depiction.

FIG. 6 is an enlarged isometric depiction of the treatment head 106. The treatment head 106 contains an optical arrangement that is capable of focusing the coherent optical energy radiation into a light beam defining a cross-sectional area in a range from about 12 square centimeters to about 20 square centimeters where the light beam emanates from the treatment head 106. That equates to the light beam at that cross section defining a diameter in a range from about four centimeters to about 5 centimeters. The novel combination of the high power laser source with the large size focused beam is what enables the present embodiments to deliver “volumetric effect” dosages of the coherent optical energy radiation at a power output from the treatment head in a range from negligibly low levels such as 0.05 watts per square centimeter up to and including about 2.0 watts per square centimeter. A laser on/off switch 174 and an actuator 176 for operating a mechanical shutter blocking the laser beam are provided on the treatment head 106 for ergonomically controlling the desired delivery of the laser beam during treatment.

The single treatment head 106 of the disclosed embodiments above is merely illustrative and not in any way limiting of the contemplated embodiments. That is, one treatment head 106 is capable of treating a finite amount of the living tissue workpiece depending on the velocity with which it is moved in accordance with a prescribed treatment protocol. In equivalent alternative embodiments two or more treatment heads 106 can each communicate the coherent optical energy radiation from the laser source 150 or even from more than one laser source in the enclosure. Simultaneous movement of multiple treatment heads 106, preferably by robotic control, increases the amount of the living tissue workpiece that can be treated in a given span of time.

Preferably, the optical arrangement in the treatment head 106 focuses the coherent optical energy radiation emitted from the treatment head to define a non-Gaussian beam energy distribution characterized by a substantially constant beam intensity across different radial positions of the beam cross-section. This non-Gaussian beam energy distribution can be generally characterized as a top hat beam being emitted from the treatment head. Preferably, also, the optical arrangement focuses the coherent optical energy radiation emitted from the treatment head to define a substantially parallel beam or even a convergent beam, instead of a divergent beam.

The foregoing FIGS. individually and collectively depict a device that is constructed in accordance with the present embodiments, contemplating a therapeutic treatment by a high level reactive laser system for the purposes of reducing pain, reducing inflammation, and enhancing healing of damaged tissue by stimulation of microcirculation, all being successfully accomplished without producing damaging thermal effects in the tissue. The disclosed diode laser is preferably used as the laser source, but any coherent light source of the preferable wavelength will work. In illustrative embodiments its principal wavelength is in the near infrared (invisible) portion of the electromagnetic spectrum at or about 1,064 nanometers, with an adjustable beam power density of 0.050 watts per square centimeter to 2.0 watts per square centimeter. The preferred operation is in continuous mode, and its output is controlled by an adjustable timer, treatment counter and power setting. Another method could use a pulsed beam. The beam is delivered to the target site by fiber optic medium and treatment head with optics assembly. The preferred beam shape range is from substantially parallel to a dynamic focusing or converging beam. Generally, the coherent optical energy radiation is controlled and applied to produce an absorption rate in the irradiated tissue which will elevate the average temperature of the irradiated tissue to a level above the basal body temperature, but without exceeding the maximum absorption rate which causes tissue overheating to the point of ablation.

A particularly advantageous feature of the present embodiments is the relatively wider beam, in a range from about 4 centimeters to about 5 centimeters and preferably about 4.4 centimeters in diameter. Those diameters correspond to a laser beam with a total exposure area emanating from the treatment head being in a range from about 12 square centimeters to about 20 square centimeters and preferably about 15.2 square centimeters. The treated living tissue is irradiated with the coherent optical energy radiation at a plurality of treatment areas concurrently or systematically in a grid for the amount of time and intensity necessary to provide a therapeutic effect, below the photoablation threshold of tissue (PAT).

It has been determined through extensive testing that the foregoing condition is satisfied by the disclosed embodiments; the diode laser being operated at its primary wavelength of 1,064 nanometers and at a power output level of from 0.050 to 2.0 watts per square centimeter. Other lasers could be used or developed to operate in a range of 950 to 1,200 nanometers and a preferred range of from about 1,000 to about 1,150 nanometers at the same power density.

The coherent optical energy radiation is applied to regions of the body which require a decrease in muscle spasm, increased circulation, decrease in pain or enhanced cellular healing. The surface area is demarcated and the surface of the living tissue is irradiated with the laser beam for the amount of time and intensity necessary to produce the desired therapeutic effect. The amount of time and intensity of treatment is determined by the character of the living tissue to be treated, the depth of penetration desired, the nature of the condition, the acuteness of the injury and the condition of the patient. In a preferred method, the amount of time is in the range from about 1 second to about 150 seconds.

Summarizing some of the key features of the present embodiments that resolve the present difficulties in the art:

1. A diode laser between 10 and 100 watts (20 W preferred) of output power.

2. A very large beam diameter 4 centimeters to 5 centimeters diameters (4.4 centimeters preferred). This allows for a large volume of energy to penetrate to the cellular level.

3. A beam energy distribution that is non-Gaussian, near flat to slightly inverse-Gaussian, with a slightly lower energy density in the middle and higher on the edges. Thus, the edges of the beam can disseminate heat more quickly—avoiding hot spot and allowing maximum energy transmission.

4. Use of longer wavelength 950 nanometer—1150 nanometer (1064 nanometer preferred) that is not easily absorbed (causing heat) by chromophores, thus allowing for deeper penetration.

5. Optics produce a parallel, cylindrical (or slightly converging) beam instead of a diverging beam. In equivalent alternative embodiments an adjustable, dynamic optics assembly is provided for selectively changing the beam shape between parallel and converging. To transmit maximum energy to a distant point (for example four inches) under the skin, a parallel or converging beam shape provides far greater energy density at that point. A parallel beam is also scattered and reflected less (meaning more forward penetration) than a diverging beam.

6. The laser is automatically self calibrating. Each treatment cycle has as a condition precedent a calibration routine that compares the observed output power level to a threshold, or expected, value. A safety interlock prevents access to the control features until the calibration routine is satisfied.

7. The laser continuously monitors power, current, and temperature for proper settings. For continuous safety, the laser system interlocks require these parameters to be within a defined range. At any time if these fall outside the expected range the control system will switch the laser into the lockout mode, requiring the calibration routine be run again before enabling the laser source.

Certain advantageous physiological mechanisms in the tissue and at the cellular level have been observed to be triggered only when the apparatus and associated process described above is employed. In the evaluation of the microcirculatory system, for example, it has been demonstrated the blood vessel walls possess photosensitivity. When the blood vessel walls are exposed to laser irradiation as set forth above, the tonus is inhibited in smooth myocytes, thus increasing the blood flow in the capillaries. Uniform effect requires saturation volume of photon energy diffusely over the treatment area. Other effects which have been observed are: peripheral capillary neovascularization, reduction of blood platelet aggregation, reduction of 0₂ from the triplet to the singlet form which allows for greater oxygenation of the tissue, reduction of buffer substance concentration in the blood, stabilization of the indices of erythrocyte deformation, reduction of products of perioxidized lipid oxygenation of the blood. Other effects which have been observed are increased index of antithrombin activity, stimulation of the enzymes of the antioxidant system such as superoxide dismutase and catalase. An increase in the venous and lymph and outflow from irradiated region has been observed. The tissue permeability in the area is substantially enhanced. This assists in the immediate reduction of edema and hematoma concentrations in the tissue. At the cellular level, the mitochondria have also been noted to produce increased amounts of ADP with subsequent increase in ATP. There also appears to be an increased stimulation of the calcium and sodium pumps at the tissue membrane at the cellular level.

At the neuronal level, the following effects have been observed as a result of the foregoing therapeutic treatment. First, there is an increased axon potential of crushed and intact nerves. The blood supply and the number of axons are increased in the irradiated area Inhibition of scar tissue is noticed when tissue is treated. There is an immediate increase in the membrane permeability of the nerve. Long term changes in the permeability of calcium and potassium ions through the nerve for at least 120 days have been observed. The RNA and subsequent DNA production is enhanced. Singlet O₂ is produced which is an important factor in cell regeneration. Pathological degeneration with nerve injury is changed to regeneration. Both astrocytes and oligodedrocytes are stimulated which causes an increased production of peripheral nerve axons and myelin.

Phagocytosis of the blood cells is increased, thereby substantially reducing infection. There also appears to be a significant anti-inflammatory phenomena which provides a decrease in the inflammation of tendons, nerves, bursae in the joints, while at the same time yielding a strengthening of collagen. There is also an effect on the significant stimulation granulation tissue in the closure of open wounds under limited circulation conditions.

Analgesia of the tissue has been observed in connection with a complex series of actions at the tissue level. At the local level, there is a vasodilation with reduction of inflammation, and a reabsorption of exudates. Enkephalins and endorphins are recruited to modulate the pain production both at the spinal cord level and in the brain. The serotonergic pathway is also recruited. While it is not completely understood, it is believed that the irradiation of the tissue causes the return of an energy balance at the cellular level.

Certain advantages of the present embodiments have also been discovered in the living tissue forming the human circulatory system. Diseased or injured cells are energy depleted and are often further compromised by poor vascular supply. It has been determined that physiologic demands may deplete cellular functional capabilities resulting in diminished cellular response in spite of available cellular capacity. Lacking compensatory energy supply, physiologic conditions may overwhelm cellular response capabilities. Accumulation of intracellular toxins impedes efficient specialized cellular function.

The high density infusion of photon energy to highly vascular areas causes a redistribution of the benefits throughout the body as the circulatory system goes. High density photon infusion to areas of compromised blood supply vasodilates blood vessels and lymphatics to improve nutrient delivery and relief from extracellular tissue fluid accumulation.

In the course of survival the central nervous and endocrine systems determine various cellular functions, subsequently the cells respond, often to the point of depleted energy reserves. Limitations of vascular flow, nutritional absorption and oxygenation determine cellular recovery from exhaustion or depletion of energy reserves. High density infrared photon saturation to a vast architecture of vascular transportation introduces energy source for cells supplementing available nutritional and oxygen sources.

The present embodiments utilize a protocol of delivery of deep penetrating dense volumes of infrared energy for local cellular absorption and use and for absorption into the vascular and electromagnetic transfer structures for secondary redistribution and ultimate delivery to individual cells. It has been determined that cells are able to utilize infrared photons as an energy source. The volumetric effect dosages of the present embodiments deliver photon energy in a way enabling large volumes of low energy or depleted energy cells to recover functional capability, regeneration, and inter and intracellular equilibrium. The volumetric effect embodiments saturate the vascular and electromagnetic redistribution delivery system at regular intervals to maintain peak function and energy reserves.

The present embodiments propose an alternative energy source for specialized cellular excretory capability combined with improved circulation to clear toxins promptly and normalize efficient cell function. This “alternative energy” preferred method would be delivered in a volumetric method to satisfy the energy needs of large masses of affected cells (muscles, organs, systems) to affect a general status quo that more closely approximates “normal” in terms of a systemic status.

It has further been determined that the present embodiments have a beneficial effect on the metabolism and evacuation of contents of fat cells. This effect is dependent on direct infusion of the infrared photons into the fat cell. The embodiments propose the volumetric massive application of energy to large areas of fat cells resulting in emptying of cellular contents into the intracellular space. The contents consist of lipid (fat) and toxins shown to be stored in fat cells. The embodiments propose improved body composition through the elimination of stored fat as well as the stimulation of underlying connective tissue and muscle cells. Again, this process must occur on a volumetric basis in order to obtain the positive impact of the present embodiments.

Infrared energy has been shown to vasodilate blood vessels and lymphatic channels, improving delivery of blood borne nutrients and pharmaceutical substances. Local vasodilation results in an effective increased exposure of treated living tissue to the nutrient and pharmaceutical concentration, when compared to non-vasodilated living tissue.

It has been determined that the present embodiments are quantifiably capable of stimulating the oxygen carrying capacity of the hemoglobin in red blood cells. The ability to significantly improve oxygen carrying capacity further assures oxygen availability to deprived cells. The volumetric effect of the infrared effect on the hemoglobin is necessary to significantly improve whole body blood volume oxygen carrying capacity and delivery. It has been determined that cells “share” a transfer energy via electromagnetic transfer. Mobile energy reserves within the bloodstream serve to balance and equilibrate the distribution of energy fuel to cells most needing it.

Other certain advantages of the present embodiments have also been discovered in the living tissue forming the human organs. The present embodiments stimulate specific cell types in organs such as the kidney, liver, pancreas, adrenals, muscle, ovaries, and testes resulting in improved specialized cell function. Stimulation of cell regeneration is not necessarily equivalent to organ hormone production as there are adequate checks and balances in the endocrine system to control levels of indigenous production. Infrared stimulation of these specialized cells and their unique cell structure serves to encourage a ready supply of fresh and efficient cell types to meet the challenges of the aging function

Through directly stimulating regeneration of specialized cell types, the infrared light applied in the parameters noted herein creates a “backup” system of efficient function cells. In this way evaluation of infrared treatment might best be considered a regeneration and cellular potential for maintaining ideal hormone and secretive enzyme values. The indigenous feedback mechanisms in the respective system then has reserve cells to convert to active productive cells and in this way maintain desirable function and filters. When applied diffusely to the tissue, all treated cells benefit equally in reversing energy deficits and return to equilibrium is modulated.

Through directly stimulating regeneration of specialized cell types, the infrared light applied in the parameters noted herein creates a “backup” system of efficient function cells. This function normalizes and stabilizes immune activity through the broad stimulation of the white blood cells and their subtypes, correcting imbalances that are characteristic of active viral and bacterial infection. The efficient production of desirable cell types and subtypes and their feedback control cell types strengthens the body's defense against organisms that neutralize specific feedback control cells to allow the organism to proliferate.

In addition to the direct stimulation of cellular regeneration, the present embodiments contemplate photon mass being absorbed and redistributed within the body so as to add a significant secondary energy source to distant fuel cell function and regeneration. Through the stimulation of systemic cellular upgrading the high energy, high density photon infusion stimulates systemic action as well as feedback control functions that control over production of hormones and secretions, cellular proliferation, cellular subtype proliferation as well as supplying an alternative energy source for these added functions to survive.

The present embodiments propose that ideally all the cells of the body contain within their structure all of the intracellular structures to carry out their genetically determined specialized function. These specialized functions require an energy source and the embodiments propose optical energy as an alternative energy source to the conventional nutrient delivery through the circulation. A threshold of energy requirements must be met to achieve the functional demands of specific environmental, chemical, and physiologic challenges to the cells, organs and system in general. Operation of cells at maximal efficient functional capacity represents the best possible scenario of “normal.” The effect of high density infrared maintenance protocol is to emulate ideal normalcy functional capacity in all specialized cell types with respect to each other and the current environmental conditions. The volumetric application of usable infrared energy overcomes the deficiency state of depleted cellular reserves and extends specialized cellular functional life and reproduction.

Although the present embodiments have been described with reference to preferred embodiments, workers skilled in the art will recognize that changes may be made in form and detail without departing from the spirit and scope of the embodiments of the present invention.

It is to be understood that even though numerous characteristics and advantages of various embodiments of the invention have been set forth in the foregoing description, together with details of the structure and function of various embodiments of the invention, this disclosure is illustrative only, and changes may be made in detail, especially in matters of structure and arrangement of parts and values for the described variables, within the principles of the present embodiments to the full extent indicated by the broad general meaning of the terms in which the appended claims are expressed.

What is claimed:

1. An apparatus for therapeutically treating living tissue, the apparatus comprising:
a laser source operably generating coherent optical energy radiation having a wavelength in a range of about 950 nanometers to about 1,200 nanometers;
an optics connector connected at a proximal end in optical communication with the laser source; and
a treatment head connected to a distal end of the optics connector, the treatment head having an optical arrangement operably focusing the coherent optical energy radiation into a light beam defining a cross-sectional area in a range from about 12 square centimeters to about 20 square centimeters where the light beam emanates from the treatment head.

2. The apparatus of claim 1 wherein the laser source operably generates the coherent optical energy radiation in a range from about 1,000 nanometers to about 1,150 nanometers.

3. The apparatus of claim 1 wherein the laser source operably generates the coherent optical energy radiation at a primary wavelength of 1,064 nanometers.

4. The apparatus of claim 1 wherein the optics connector is a flexible fiber optics device capable of maintaining the optical communication between the laser source and the treatment head during a time when the treatment head is being selectively moved in relation to the laser source.

5. The apparatus of claim 1 further comprising calibration logic that calibrates a power level of the coherent optical energy radiation emitted by the treatment head in relation to a selected power level as a condition precedent to enabling the treatment head to operably emit the coherent optical energy radiation at the selected power level.

6. The apparatus of claim 5 further comprising:
a cradle sized to receivingly engage a distal end of the treatment head;
an interlock in the cradle sensing when the treatment head is in the cradle;
a power sensing device in communication with the treatment head when the treatment head is diposed in the cradle; and
computer instructions stored in memory and executed to perform operational steps of the calibration logic including comparing an observed power level from the treatment head to a threshold power level.

7. The apparatus of claim 1 wherein the optical arrangement operably focuses the coherent optical energy radiation emitted from the treatment head to define a non-Gaussian beam energy distribution characterized by a substantially constant beam intensity across different radial positions of the beam cross-section.

8. The apparatus of claim 7 wherein the optical arrangement operably focuses the coherent optical energy radiation emitted from the treatment head to define a substantially parallel beam energy distribution.

9. The apparatus of claim 7 wherein the optical arrangement operably focuses the coherent optical energy radiation emitted from the treatment head to define a substantially converging beam energy distribution.

10. The apparatus of claim 1 wherein the laser source is selectable to provide the coherent optical energy radiation at a power level in a range from about 10 Watts to about 100 Watts.

11. The apparatus of claim 1 wherein the laser source is selectable to provide the coherent optical energy radiation at a maximum power level of about 20 Watts.

12. The apparatus of claim 1 comprising a plurality of treatment heads, each of the plurality connected to a distal end of a respective optics connector and having a respective optical arrangement operably focusing the coherent optical energy radiation into a light beam defining a cross-sectional area in the range from about 12 square centimeters to about 20 square centimeters where the light beam emanates from the treatment head.

13. A therapeutic laser device comprising a laser source operably generating coherent optical energy radiation having a wavelength in a range of about 950 nanometers to about 1,200 nanometers and at a power output from a treatment head in a range from about 0.05 watts per square centimeter to about 2.0 watts per square centimeter.

14. The therapeutic laser device of claim 13 wherein the treatment head emits the coherent optical energy radiation defining a cross-sectional area in a range from about 12 square centimeters to about 20 square centimeters.

15. The therapeutic laser device of claim 13 further comprising calibration logic that enables the treatment head to operably emit the coherent optical energy radiation only after calibrating the power level of the coherent optical energy radiation.

16. The therapeutic laser device of claim 13 wherein the treatment head operably focuses the coherent optical energy radiation emitted from the treatment head to define a non-Gaussian beam energy distribution characterized by a substantially constant beam intensity across different radial positions of the beam cross-section.

17. The therapeutic laser device of claim 13 wherein the laser source is selectable to provide the coherent optical energy radiation at a power level in a range from about 1 Watt to about 100 Watts.

18. The apparatus of claim 13 wherein the treatment head first emits the coherent optical energy radiation defining a cross-sectional diameter in a range from about four centimeters to about five centimeters.

19. A method comprising:
obtaining an apparatus having a laser source operably generating coherent optical energy radiation with a wavelength in a range of about 950 nanometers to about 1,200 nanometers, an optics connector connected at a proximal end in optical communication with the laser source, and one or more treatment heads each connected to a distal end of a respective one of the optics connector, each treatment head operably focusing the coherent optical energy radiation into a light beam defining a cross-sectional area in a range from about 12 square centimeters to about 20 square centimeters where the light beam emanates from the treatment head;
aiming the treatment head to irradiate a selected live tissue workpiece with the coherent optical energy radiation; and
controlling a dwell time that the selected live tissue workpiece is irradiated in accordance with a predefined treatment protocol.

20. The method of claim 19 further comprising, before the aiming feature, calibrating a power of the coherent optical energy radiation emitted from the treatment head by:
selecting a desired power level of the coherent optical energy radiation;
monitoring an interlock to determine whether the treatment head is disposed in a calibration position;
when the monitoring indicates the treatment head is disposed in a calibration position, measuring a power level of the coherent optical energy radiation emitted from the treatment head;
comparing the results of the monitoring to the selected power level; and
only if the results of the comparing is less than a predetermined threshold value, then enabling the treatment head to emit the coherent optical energy radiation.
